(19) United States
(12) Patent Application Publication
(10) Pub. No.: US 2008/0195141 A1
(43) Pub. Date: Aug. 14, 2008

(54) BACKSTOP PROTECTION DEVICE AND METHOD OF USING THE SAME

(76) Inventor: James Teague, Spencer, IN (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 12/027,449

(22) Filed: Feb. 7, 2008

Related U.S. Application Data
(60) Provisional application No. 60/888,892, filed on Feb. 8, 2007.

(51) Int. Cl. A61M 29/00

(52) U.S. Cl. 606/200

(57) ABSTRACT

An apparatus according to an embodiment of the invention includes an elongate body disposable within a body lumen of a patient. An expandable member is coupled to the elongate body. The expandable member has a collapsed configuration and an expanded configuration. The expandable member when in the expanded configuration is configured to substantially prevent distal migration of biological material within the body lumen during a medical procedure being performed in the body lumen using a medical device different than the elongate body. In another embodiment, an apparatus includes an expandable member having a collapsed configuration and an expanded configuration and a membrane is coupled to the expandable member. The membrane is configured and formulated to maintain adequate structural integrity when exposed to laser energy applied during a lithotripsy procedure performed within a body lumen when the expandable member is disposed within the body lumen.

### CROSS-REFERENCE TO RELATED APPLICATIONS

This application claims priority to U.S. Provisional Application No. 60/888,892, entitled “Backstop Protection Device and Method of Using the Same,” filed Feb. 8, 2007, the disclosure of which is hereby incorporated by reference in its entirety.

### BACKGROUND

The disclosed invention relates generally to a medical device and more particularly to a medical device that can be used as a protective backstop or a retention device during a medical procedure.

A variety of medical procedures are performed within various body lumens of a patient, such as a lithotripsy procedure within a ureter (to break-up kidney stones), or a vascular procedure within a vascular lumen. During such medical procedures, biological material, such as blood clots (thrombi) during a vascular procedure, or portions of a kidney stone during a lithotripsy procedure, can migrate distally within the body lumen resulting in potentially dangerous consequences for the patient. For example, a thrombus can migrate distally within a vascular lumen during a vascular procedure and cause an embolism (blockage of a vascular lumen).

Some medical devices may provide a protective backstop to inhibit distal migration, however, known devices may not be suitable for use in connection with a lithotripsy procedure. One problem is that the laser used for the lithotripsy may damage the backstop. Another problem is that the devices that may be used as a backstop are typically relatively large, such that the device must occupy the working channel of the endoscope in lieu of other devices, requiring the sequential insertion of devices in the working channel.

Thus, it would be desirable to have a device that can be used as a backstop to prevent distal migration of biological material within a body lumen that is constructed with a material resistant to energy emitted by a laser, and/or sized such that the device can fit within the body lumen (without occupying the working channel of an endoscope) during a medical procedure being performed by another medical device.

### SUMMARY OF THE INVENTION

An apparatus according to an embodiment of the invention includes an elongate body disposable within a body lumen of a patient. An expandable member is coupled to the elongate body. The expandable member has a collapsed configuration and an expanded configuration. The expandable member when in the expanded configuration is configured to substantially prevent distal migration of biological material within the body lumen during a medical procedure being performed in the body lumen using a medical device different than the elongate body. In another embodiment, an apparatus includes an expandable member having a collapsed configuration and an expanded configuration and a membrane is coupled to the expandable member. The membrane is configured and formulated to maintain adequate structural integrity when exposed to laser energy applied during a lithotripsy procedure performed within a body lumen when the expandable member is disposed within the body lumen.

### BRIEF DESCRIPTION OF THE DRAWINGS

FIG. 1 is a schematic illustration of a medical device according to an embodiment of the invention.

FIGS. 2 and 3 are side views of a medical device according to an embodiment of the invention shown in a collapsed configuration and an expanded configuration, respectively.

FIG. 4 is a side cross-sectional view of the medical device of FIGS. 2 and 3, taken along line 4-4 in FIG. 2.

FIG. 5 is a cross-sectional view of the medical device of FIGS. 2 and 3, taken along line 5-5 in FIG. 2.

FIG. 6 is a side cross-sectional view of the medical device of FIGS. 2 and 3 taken along line 6-6 in FIG. 3.

FIG. 7 is a cross-sectional view of the medical device of FIGS. 2 and 3 taken along line 7-7 in FIG. 3.

FIG. 8 is a side view of the medical device of FIGS. 2 and 3 shown disposed within a ureter in an expanded configuration.

FIG. 9 is a side view of a medical device according to another embodiment of the invention shown in an expanded configuration.

FIG. 10 is a cross-sectional view of the medical device of FIG. 9 taken along line 10-10 in FIG. 9.

FIG. 11 is a side view of the medical device of FIG. 9 shown in a collapsed configuration.

FIG. 12 is a side perspective view of a portion of the medical device of FIG. 9 shown in a partially expanded configuration.

FIG. 13 is an end perspective view of the portion of the medical device of FIG. 9 shown in a partially expanded configuration.

FIG. 14 is a side view of a medical device according to another embodiment of the invention shown in an expanded configuration.

FIG. 15 is a cross-sectional view of the medical device of FIG. 14 taken along line 15-15 in FIG. 14.

FIGS. 16 and 17 are each side views of the medical device of FIGS. 14 and 15 shown in an expanded configuration and disposed within a ureter.

FIG. 18 is a side view of the medical device of FIGS. 14 and 15 shown in an expanded configuration and disposed within a vascular lumen.

FIG. 19 is a side view of a medical device according to yet another embodiment of the invention shown in an expanded configuration.

FIG. 20 is a cross-sectional view taken along line 20-20 in FIG. 19.

FIG. 21 is a cross-sectional view of a medical device according to another embodiment of the invention.

FIG. 22 is a flowchart of a method according to an embodiment of the invention.

FIG. 23 is a side view of the expandable member of FIGS. 2 and 3 shown in a collapsed configuration.

FIG. 24 is a cross-sectional view taken along line 24-24 in FIG. 23.

### DETAILED DESCRIPTION

An apparatus according to an embodiment of the invention is configured to be used during a medical procedure being performed by another medical device within a body lumen. A medical device is provided that can perform a variety of functions. For example, a medical device according to an embodiment of the invention can be used as a guide wire for guiding other devices within a body lumen of a patient, or as a backstop device during a procedure such as a lithotripsy procedure in a ureter, or as a protection device during a procedure being performed within a vascular lumen.

In one embodiment, an apparatus includes an elongate body disposable within a body lumen of a patient. An expandable member is coupled to the elongate body. The expandable member has a collapsed configuration and an expanded configuration. The expandable member when in the expanded configuration is configured to substantially prevent distal migration of biological material within the body lumen during a medical procedure being performed in the body lumen using a medical device different than the elongate body. In some embodiments, the expandable member is embodied in part as a cannula or tubular member. In some embodiments, another cannula or tubular member can be used to actuate or move the expandable member between the collapsed configuration and the expanded configuration.

As used herein, the term proximal refers to the portion or end of a device that is closest to a physician when performing a medical procedure, and the term distal refers to the portion or end of the device that is furthest from the physician during a medical procedure. For example, a distal end or portion of a medical device as described herein refers to the end or portion of the medical device that is first inserted into a body of a patient during a medical procedure. The proximal end or portion is the end or portion of the medical device that is inserted into a body of the patient after the distal end or portion.

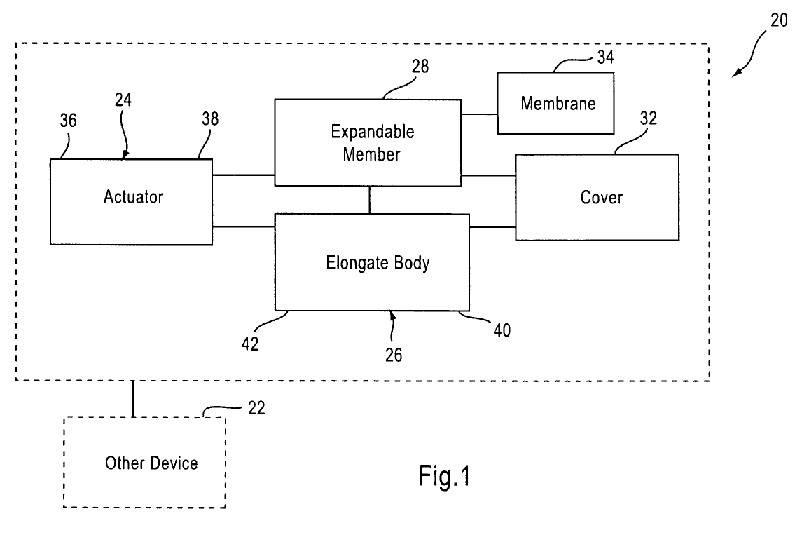

FIG. 1 is a schematic illustration of a medical device according to an embodiment of the invention. A medical device 20 is configured to be placed or otherwise inserted into a body lumen of a patient (not shown in FIG. 1) for use in conjunction with a second medical device 22 to be used to perform a medical procedure within the body lumen. The medical device 20 can be coupled to the second medical device 22, or provided as a detached separate device. The medical device 20 includes an elongate body 26 having a proximal portion 42 and a distal portion 40. The elongate body 26 can be constructed, for example, in the same manner as, and be capable of performing the guiding function of, a guide wire and/or as a safety wire to maintain access to, for example, a ureter.

An expandable member 28 can be disposed at the distal portion 40 of the elongate body 26. The expandable member 28 can be moved between a collapsed configuration, in which the medical deice 20 can be inserted into a body lumen of a patient (and optionally can pass through the working channel of a medical device, such as an endoscope), and an expanded configuration, in which the expandable member 28 can retain the medical device 20 within the body lumen of the patient and/or serve as a protective backstop, and/or serve to sweep biologic debris from the body lumen. The expandable member 28 defines an outer perimeter (not shown in FIG. 1) when the expandable member 28 is in the expanded configuration that is greater than an outer perimeter defined by the expandable member 28 when the expandable member 28 is in the collapsed configuration. The expandable member 28 can be a variety of different configurations when in the expanded configuration, such as having a substantially circular perimeter, a square perimeter shape, a hexagonal perimeter, etc. When the expandable member 28 is in the expanded configuration, the medical device 20 can be used for a variety of purposes, such as a guide wire, and/or a protective backstop and/or to sweep biological material from a body lumen.

The proximal portion 42 of the elongate body 26 can be coupled to a distal portion 38 of an actuator 24. The actuator 24, can be, for example, in the form of a pusher cannula. For example, the actuator 24 can be placed over the proximal portion 42 of the elongate body 26 such that a distal end of the actuator 24 contacts a proximal end of the expandable member 28 or a proximal end of another component, such as a cover member or cannula that is coupled to the expandable member 28. The actuator 24 can be used to move the expandable member 28 between the collapsed configuration and the expanded configuration, as will be described in more detail below with reference to specific embodiments.

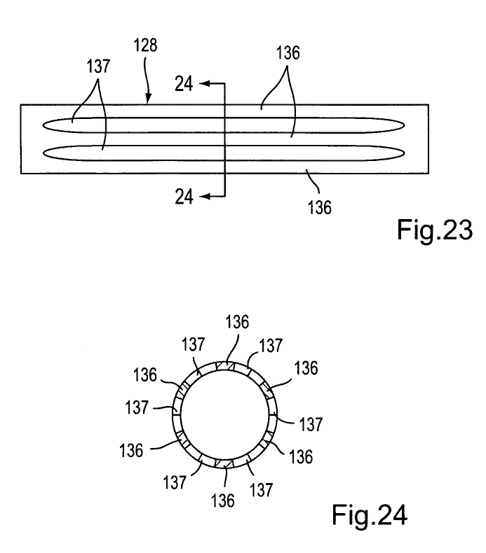

The expandable member 28 can include one or more deformable portions (not shown in FIG. 1). For example, the expandable member 28 can include one or more wire members (not shown in FIG. 1) coupled to a cannula or cover member in which the elongate body 26 can be movably disposed. Such a cannula or cover member can be coupled to the actuator 24 or the actuator 24 can be placed in contact with the cannula or cover member as mentioned above. In alternative embodiments, the deformable portions of the expandable member 28 can be embodied in an expandable tubular member, such as a cannula, that can be coupled to the elongate body 26 (e.g., the elongate body 26 can be movably disposed within a lumen of the expandable member). For example, a cannula can be formed from a tubing material and one or more deformable portions can be formed by laser cutting a sidewall of the tubing. Such an embodiment is described in more detail below with reference to FIGS. 23 and 24. In such an embodiment, the expandable member 28 can also be coupled to the actuator 24 or configured to be engaged by the actuator 24. The expandable member 28 can define an interior volume when the expandable member 28 is in both the collapsed and expanded configurations that can be substantially closed with, for example, a membrane as described below.

The medical device 20 can also include a membrane 34 that can be coupled, for example, to the expandable member 28. For example, the membrane 34 can be coupled to and substantially covering the deformable portion(s) of the expandable member 28. The membrane 34 can be formed with a material that is resistant to laser energy of the wavelength, intensity, duty cycle, and duration used during, for example, a laser lithotripsy procedure, to protect the membrane 34 from damage. In some embodiments, the membrane 34 can be formed with an expandable material and be configured as, for example, a tubular member coupled over the expandable member 28. In such an embodiment the membrane 34 can expand as the expandable member is moved from the collapsed configuration to the expanded configuration. The membrane 34 can in some embodiments be permeable. For example, the membrane 34 can be formed with a mesh material having one or more open portions.

A cover member 32 can be coupled to the distal portion 40 of the elongate body 26. The medical device 20 can also include a second cover member (not shown in FIG. 1) coupled to the proximal portion 42 of the elongate body 26. The cover member 32 can also be coupled to a portion of the expandable member 28. The cover member 32 can be formed with, for example, a polytetrafluoroethylene (PTFE) or hydrophylically coated polyurethane material, which provides for easier insertion of the medical device into the body lumen of the patient. The cover member 32 can also help prevent biological material from becoming embedded within portions of the medical device when inserted into a body lumen. The cover member 32 can be various lengths such that it extends distally from the expandable member 28 at various distances.

A distal tip (not shown in FIG. 1) can also be included on a distal end of the elongate body 26. The distal tip can be formed, for example, with a polyurethane material and can be curved or rounded to further assist with insertion of the medical device into a body lumen. The distal tip can also be coupled to the expandable member 28.

The medical device 20 can be inserted into a body lumen of a patient with the expandable member 28 in the collapsed configuration, and then reconfigured to the expanded configuration at a desired location within the body lumen. The medical device 20 can be used to perform a variety of functions while disposed within a body lumen of a patient and with the expandable member 28 in the expanded configuration. For example, the medical device 20 can be used as a guide wire with the expandable member 28 configured to help retain the medical device 20 within a body lumen of the patient. In a similar use, the medical device 20 can be used as a safety wire to maintain access to the body lumen of the patient. In such uses, a membrane 34 may not be required as the deformable portions of the expandable member 28 can contact the walls of the body lumen to retain the medical device 20 in the selected position within the body lumen.

The medical device 20 can also be used as a backstop device during, for example, a lithotripsy procedure (to break up kidney stones) in a ureter of a patient. In this use, the expandable member 28 can be used as a backstop to a kidney stone within the ureter and prevent biological material, such as kidney stone chips, from migrating distally within the body lumen as the lithotripsy procedure is being performed. As stated above, the membrane 34 can be formed with a material sufficiently resistant to the laser energy encountered in a lithotripsy procedure while maintaining adequate structural integrity during the procedure. Also during a lithotripsy procedure, the medical device 20 can be used to sweep the ureter to capture loose kidney stone chips with the membrane 34.

The medical device 20 can also be used as a protective device during, for example, a vascular procedure. In such a use, the expandable member 28 and the membrane 34 can prevent, for example, thrombi from migrating distally within the vascular lumen.

Thus, as mentioned previously, the medical device 20 can be sized and configured to be used in conjunction with a second medical device 22, such as serving as a guide wire for the second medical device, while the second medical device is performing a medical procedure within a body lumen. For example, the elongate body 26 can have an outer diameter as small as, for example, 0.021″ (0.533 mm) or have a maximum outer diameter typical of guide wires, such as, for example, 0.038 inches (0.965 mm) when the expandable member 28 is in its collapsed configuration. This can allow the medical device 20 to be disposed in a guide wire channel of the second medical device, or to be disposed alongside the second medical device in the body lumen. For example, an inner diameter of a typical ureter can be, for example, 0.197″ (5 mm) to 0.354″ (9 mm), and a typical endoscope can have an outer diameter sized and configured to be inserted into the ureter. Such an endoscope can have a lumen for passing a guidewire therethrough with an inner diameter, for example, of approximately 0.146″ (3.7 mm) to 0.177″ (4.5 mm). Thus, the elongate body 26 having an outer diameter of 0.021″ (0.533 mm) to 0.038″ (0.965 mm) can be inserted through a lumen of an endoscope and/or inserted directly into a ureter with sufficient space remaining within the ureter to insert a second medical device to perform a procedure within the ureter.

Having described above various general principles, several exemplary embodiments of these concepts are now described. These embodiments are only examples, and many other configurations of medical device 20 and its various components are contemplated by the principles of the invention, and will be apparent to the artisan in view of the general principles described above and the exemplary embodiments.

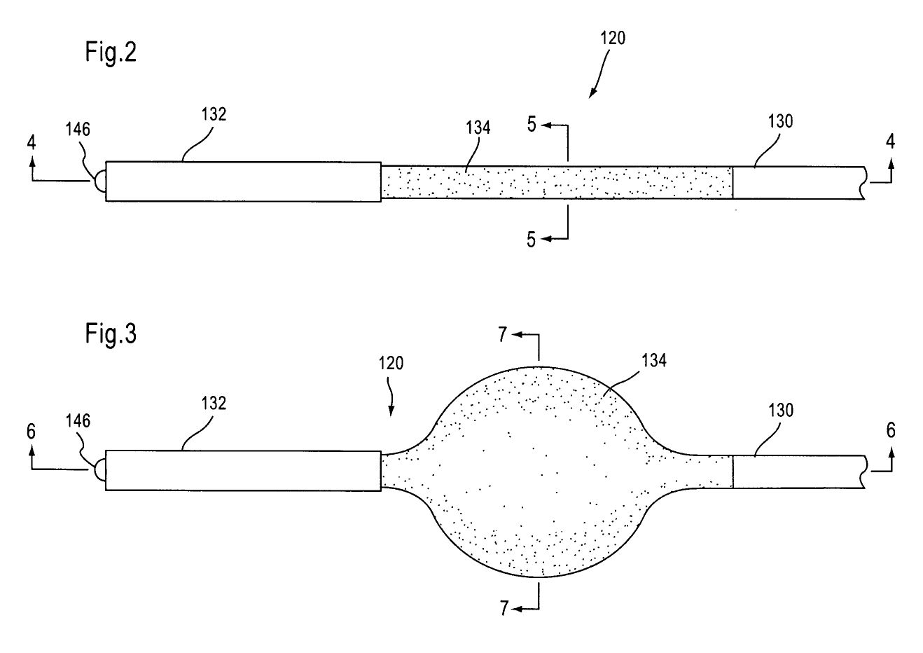

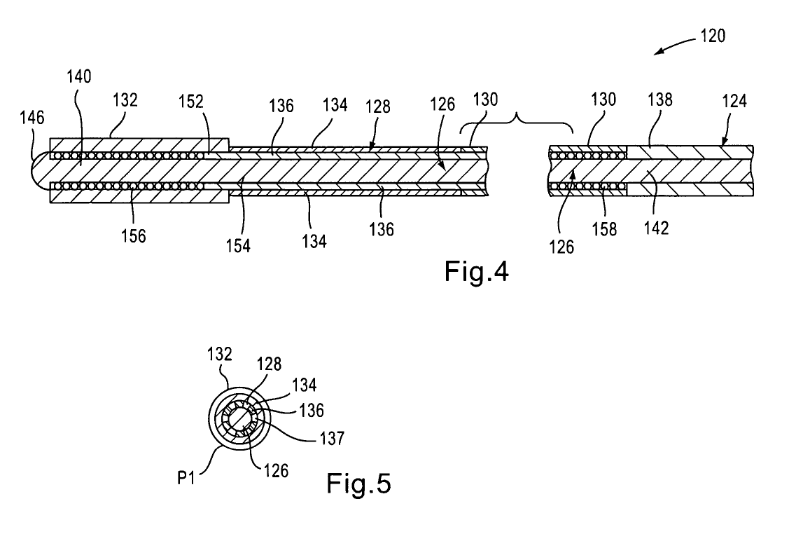

FIGS. 2-7 illustrate a portion of a medical device according to an embodiment of the invention. A medical device 120 includes an elongate body 126 (shown in FIGS. 4-7), an expandable member 128 (shown in FIGS. 4-7) and an actuator 124. In this embodiment, the expandable member 128 is formed as a cylindrical tube and includes six deformable portions 136 defined by six slots 137 (see FIGS. 23 and 24) that extend through, and axially along, the side wall of the expandable member 128. The slots 137 may be formed by any suitable technique, such as by laser cutting. The medical device 120 also includes a membrane 134 disposed over, and substantially covering the deformable portions 136 of the expandable member 128.

The elongate body 126 is movably disposable within a lumen defined by the expandable member 128 and movably disposable within a lumen defined by the actuator 124 as shown in FIGS. 4-7. A first coil member 156 is disposed at a distal portion 140 of the elongate body 126 and a second coil member 158 is disposed at a proximal portion 142 of the elongate body 126. The elongate body 126 includes a core portion 154 and a distal tip 146. The core portion 154 can be, for example, in the form of a tapered mandrel. The coil members 156 and 158 each define a lumen (not shown) in which the core portion 154 can be received such that the core portion 154 can move relative to the coil members 156 and 158. The coil members 156 and 158 can add stability and provide kink resistance to the elongate body 126 when maneuvering the elongate body 126 within a body lumen of a patient. In some embodiments, the coil members 156 and 158 can also help bias the expandable member 128 into a collapsible configuration as will be described in more detail below.

The distal tip 146 of the elongate body 126 can be coupled to a cover member 132. The cover member 132 is configured to fit over the coil portion 156. In some embodiments, the cover member 132 is coupled to the elongate body 126 such that a portion of the core 154 of the elongate body extends distally beyond the cover member 132. The cover member 132 is shown as having a particular length in FIGS. 2, 4 and 6 for illustrative purposes, but it should be understood that the cover member 132 can be longer or shorter as desired. The cover member 132 can alternatively, or additionally, be coupled to a distal portion 152 of the expandable member 128 with, for example, a laser weld or other suitable coupling methods.

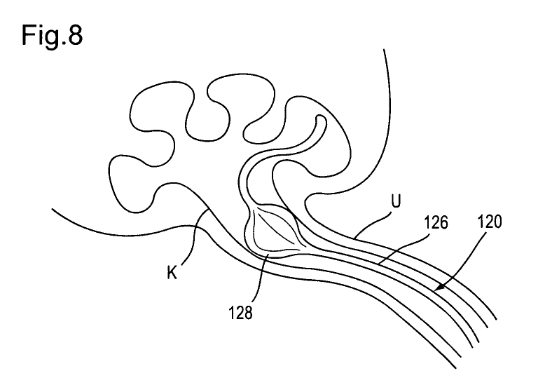

A cannula or cover member 130 is coupled to a proximal portion 150 of the expandable member 128 (e.g., with a laser weld or other suitable coupling method) and defines a lumen in which the elongate body 126 is partially disposed. The cover member 130 also extends over the coil member 158 to prevent biological material from intruding into the coil member 158. A proximal end of the cover member 130 can be coupled to a distal portion 138 of the actuator 124 or the actuator 24 can be configured to be slid over the elongate body 126 and engage the cover member 130 as needed and used to actuate the medical device 120. The cover member 130 can have a variety of different lengths. For example, the cover member 130 can have a length such that it can extend through a body lumen of a patient, such as a ureter (shown in FIG. 8) and the actuator 24 can extend outside of the patient's body.

In some embodiments, the proximal portion 150 of the expandable member 128 can extend over the coil member 158. In such an embodiment, the cover member 130 may not be included. For example, the proximal portion 150 of the expandable member 128 can extend over the coil member 158 and be coupled to a distal portion 138 of the actuator 124 (or can be configured to be engaged by the actuator 124). In some embodiments, the medical device 120 does not include the coil member 156 and/or the coil member 158.

The expandable member 128 has a collapsed configuration, as shown in FIGS. 2, 4 and 5, and an expanded configuration, as shown in FIGS. 3, 6 and 7. When the expandable member 128 is in the collapsed configuration, the medical device 120 defines an outer perimeter P1, as shown in FIG. 5, that is less than an outer perimeter P2 defined by the medical device 120 when the expandable member 128 is in the expanded configuration, as shown in FIG. 7.

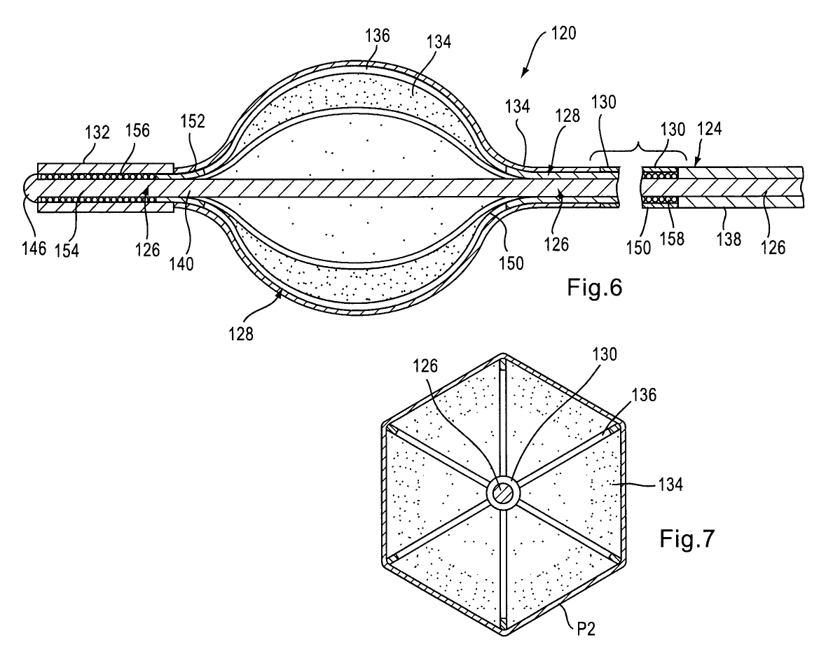

In use, the medical device 120 can be inserted into a body lumen of a patient with the expandable member 128 in the collapsed configuration, as shown in FIGS. 2, 4 and 5. The coil members 156 and 158 can help bias the expandable member 128 into the collapsed configuration. The proximal portion 142 of the elongate body 126 can be used to maneuver the distal portion 140 of the elongate body 126 to a desired location within the body lumen. Once the medical device 120 is at the desired location, the actuator 124 can be displaced distally (e.g., pushed distally) over a portion of the elongate body 126 while holding the elongate body 126 stationary. A force is then imparted by the actuator 124 (in a distal direction) on the coil member 158 (and cover member 130) and the proximal portion 150 of the expandable member 128, which causes the expandable member 128 to move to the expanded configuration, as shown in FIGS. 3, 6 and 7. In this embodiment, the expandable member 128 when in the expanded configuration has a hexagonal outer perimeter shape, as shown in the cross-sectional view of FIG. 7, and a substantially rounded shape in a side view, as shown in FIGS. 3 and 6.

In the expanded configuration, the medical device 120 can be used for various purposes as described previously. For example, FIG. 8 illustrates the medical device 120 positioned within a ureter U near an opening to a kidney K. The expandable member 128 (concealed by the membrane 134 as shown in FIG. 8) helps retain the medical device 120 in position and can be used as a guide wire for insertion of other medical devices into the ureter U. Because an outer diameter or an outer perimeter of the medical device 120 (e.g., the elongate body 126, cover member 132 and cover member 130) is sufficiently small at portions of the medical device 120 other than where the expandable member 128 is located, a second device (not shown) can be inserted into the ureter U. The medical device 120 can also be used as a backstop device during a medical procedure being performed by another device within the ureter U.

After the desired medical procedure has been performed, the expandable member 128 can be moved back to the collapsed configuration for removal of the medical device 120 from the body lumen. To move the expandable member 128 from the expanded configuration back to the collapsed configuration, the actuator 124 can be displaced proximally (e.g., pulled proximally) over the elongate body 126 while holding the elongate body 126 stationary. This action releases the force imparted on the coil member 158 and the expandable member 128, and moves the expandable member 128 to the collapsed configuration.

An alternative method of moving the expandable member 128 between the collapsed configuration and the expanded configuration is to move the elongate body 126 relative to the actuator 124. For example, to move the expandable member 128 from the expandable configuration to the collapsed configuration, the proximal portion 142 of the elongate body 126 can be pulled proximally while holding the actuator 124 stationary. This action causes the coil member 156 to compress and impart a force on the distal portion 152 of the expandable member 128, which in turn moves the expandable member 128 to the expanded configuration. To move the expandable member 128 from the expanded configuration to the collapsed configuration, the proximal portion 142 of the elongate body 126 can be moved distally while holding the actuator 124 stationary, which releases the force imparted on the coiled member 156 and the expandable member 128.

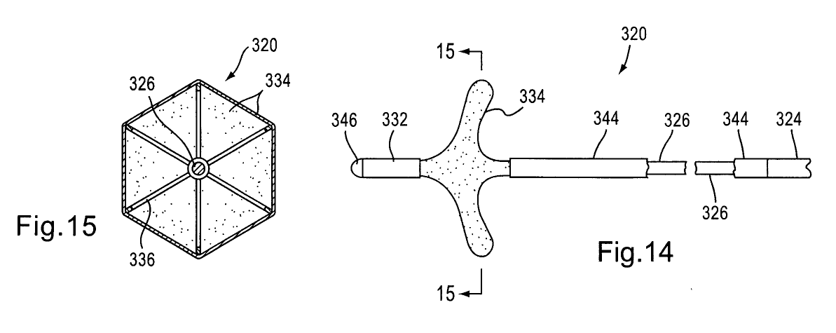

In some embodiments, the expandable member 128 can be expanded further by imparting greater force when moving the expandable member 128 to the expanded configuration. Further expansion of the expandable member 128 can result in the expandable member 128 having a shape similar to the embodiment illustrated in FIG. 14. In other words, a distal portion of the expandable member 128 can be forced to fold or collapse partially over a proximal portion of the expandable member 128 when sufficient force is imparted with the actuator 124.

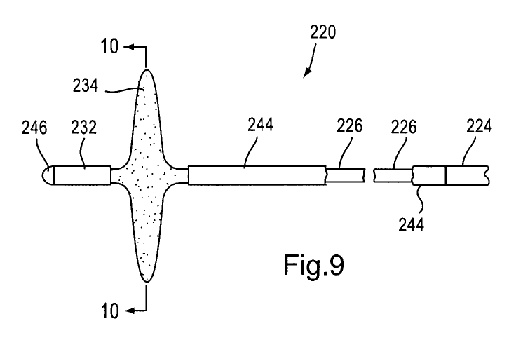

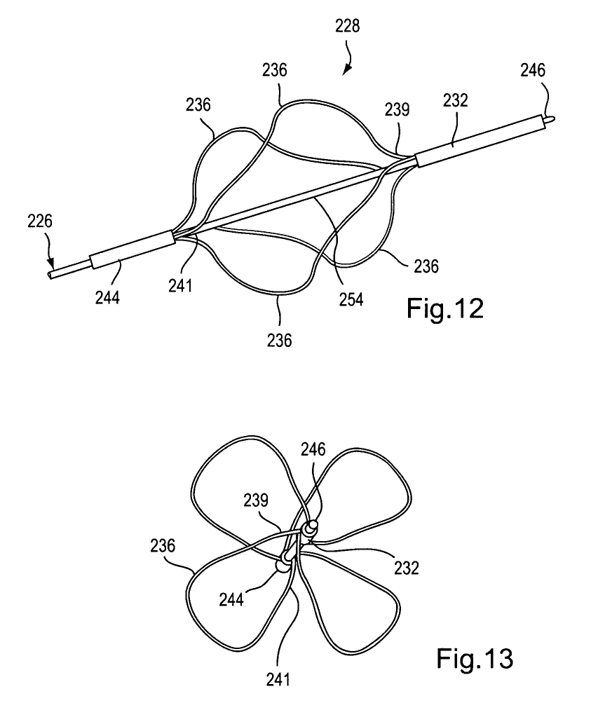

FIGS. 9-13 illustrate a medical device according to another embodiment of the invention. A medical device 220 includes an elongate body 226 movably disposable within a lumen (not shown) defined by an actuator 224. An expandable member 228 is disposed at a distal portion of the elongate body 226. In this embodiment, the expandable member 228 includes four deformable wire portions 236 coupled to a cover member 232 and a cover member 244, and a membrane 234 coupled to the deformable wire portions 236. As with the previous embodiment, the elongate body 226 includes a core portion 254 (see FIG. 12) and a distal tip 246.

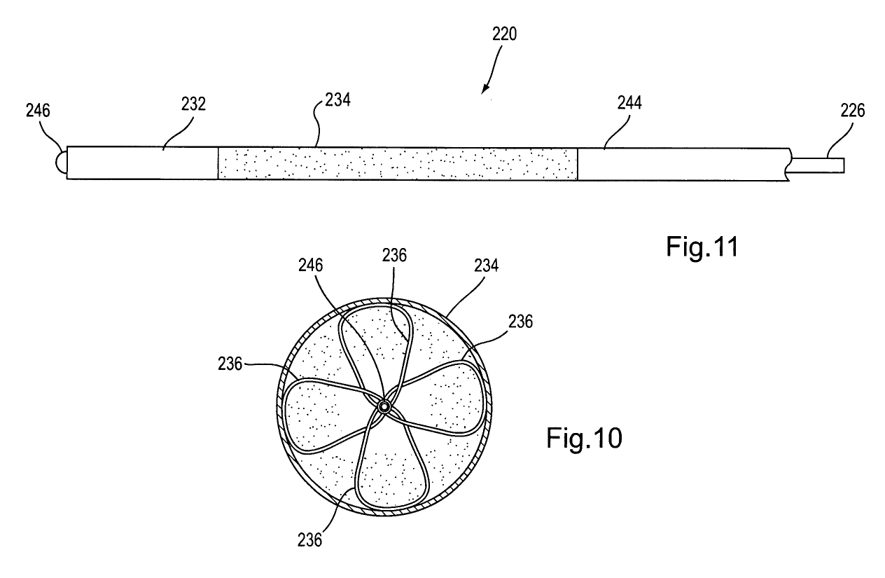

FIG. 9 illustrates the expandable member 228 in an expanded configuration and FIG. 11 illustrates the expandable member 228 in a collapsed configuration. FIG. 10 is a cross-sectional view of the medical device 220 in the expanded configuration. In this embodiment, the expandable member 228 has a helical or coiled configuration. The medical device 220 can perform any of the same functions as described previously. For example, in the expanded configuration, the medical device 220 can be used as a backstop within a body lumen, and the membrane 234 can prevent biological material from migrating distally within the body lumen.

FIG. 12 is a side perspective view of a portion of the medical device 220 illustrating the deformable wire portions 236 of the expandable member 228 (without the membrane 234) coupled to the elongate body 226, and in a partially expanded configuration. FIG. 13 is an end perspective view of the portion of the medical device 220 shown in FIG. 12. As with the previous embodiment, the medical device 220 can optionally include a first coil member (not shown) coupled to the elongate body 226 at a distal end of the expandable member 228 and a second coil member (not shown) coupled to the elongate body 226 at a proximal end of the elongate body 226. A first cover member 232 and a second cover member 244 are each coupled to the elongate body 226 as shown in FIGS. 9 and 11-13. If the medical device includes coil members, the cover members 232, 244 can be disposed over the coil members. The second cover member 244 is coupled to the elongate body 226 such that the cover member 244 can be displaced over the elongate body 226 (e.g., pushed distally by the actuator 224) to actuate the expandable member 228 between its expanded and collapsed configurations. For example the elongate body 226 can be disposed within a lumen (not shown) of the cover member 232 and a lumen (not shown) of the cover member 244.

As shown in FIGS. 12 and 13, the deformable wire portions 236 have a first end (or distal end) 239 and a second end (or proximal end) 241. In this embodiment, the first end 239 is coupled to the first cover member 232 at a first location, and the second end 241 is coupled to the second cover member 244 at a second location that is rotated approximately 90° from the first location. This provides the deformable wire portions 236 with a partial coiled or semi-helical configuration. The deformable wire portions 236 can alternatively be coupled to the coil members if coil members are included. As stated above, FIG. 13 illustrates the expandable member 228 in a partial expanded configuration. When the expandable member 228 is in a fully expanded configuration, as shown in FIG. 9, the first end 239 and the second end 241 of the deformable wire portions 236 are moved to a closer position relative to each other than the position shown in FIG. 13.

The actuator 224 can be coupled to the cover member 244 and used to move the expandable member 228 between the collapsed configuration and the expanded configuration in a similar manner as described in previous embodiments. For example, to move the expandable member 228 from the collapsed configuration to the expanded configuration, the actuator 224 can be moved distally (e.g., pushed distally) while holding a proximal end of the elongate body 226 stationary such that the cover member 244 is moved distally. This action in turn pushes the second ends 241 of the deformable wire portions 236 distally causing the expandable member 228 to move to the expanded configuration.

FIGS. 14-15 illustrate a medical device according to another embodiment of the invention. A medical device 320 includes an actuator 324 and an elongate body 326 movably disposable within a lumen (not shown) of the actuator 324. An expandable member 328 is disposed at a distal portion of the elongate body 326. In this embodiment, the expandable member 328 includes six deformable wire portions 336 and a membrane 334 coupled to the deformable wire portions 336.

The medical device 320 can also include one or more coiled members (not shown), and the elongate body 326 can include a core portion (not shown) and a distal tip 346 as described above for other embodiments. A first cover member 332 can be coupled to the elongate body 326 at a distal end of the expandable member 328, and a second cover member 344 can be coupled to the elongate body 326 at a proximal end of the expandable member 328. For example, the elongate body 326 is movably disposed within a lumen defined by the first cover member 332 and a lumen defined by the second cover member 344 as shown in FIG. 14. The deformable wire portions 336 can be coupled to the cover members 332 and 344 in a similar manner as described in the previous embodiment. In this embodiment, the deformable wire portions 336 do not form a semi-helical or partially coiled configuration (as shown in FIG. 15). In some embodiments, the deformable wire portions can be coupled to the coil members (if included in the medical device).

The expandable member 328 has a collapsed configuration (not shown), and an expanded configuration, as shown in FIGS. 14. The actuator 324 can move the expandable member 328 between the collapsed configuration and the expanded configuration in the same manner as described for previous embodiments. In this embodiment, the expandable member 328 has an expanded configuration having a substantially hexagonal outer perimeter shape, as shown in the cross-sectional view of FIG. 15, and an umbrella shape as shown in the side view of FIG. 14. The medical device 320 can perform the same functions as described for previous embodiments.

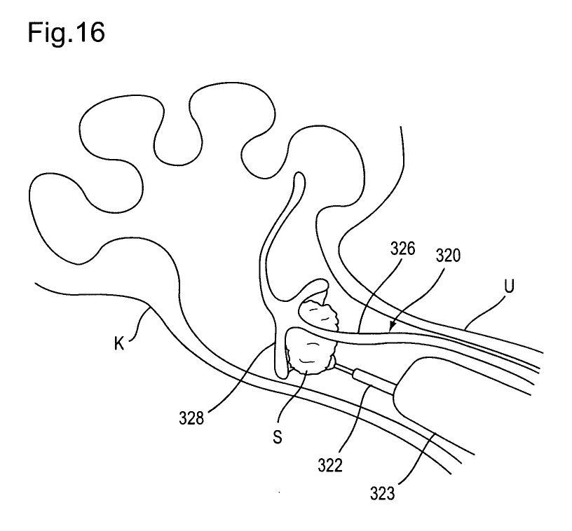

FIG. 16 is a side view of a portion of the medical device 320 shown in an expanded configuration and disposed within a ureter U near an opening of a kidney K. FIG. 16 illustrates the use of medical device 320 as a backstop during a lithotripsy procedure to break a kidney stone S into smaller portions. A lithotripsy device 322 is shown inserted through an endoscope 323 positioned within the ureter U. In some embodiments, the medical device 320 can also be inserted through a lumen of the endoscope, instead of along side the endoscope 323, as shown in FIG. 16. As illustrated in FIG. 16, an outer diameter or outer perimeter of the medical device 320 (e.g., the elongate body 326 and cover member 344) is sufficiently small to allow the medical device 320 to be used together with the lithotripsy device 322.

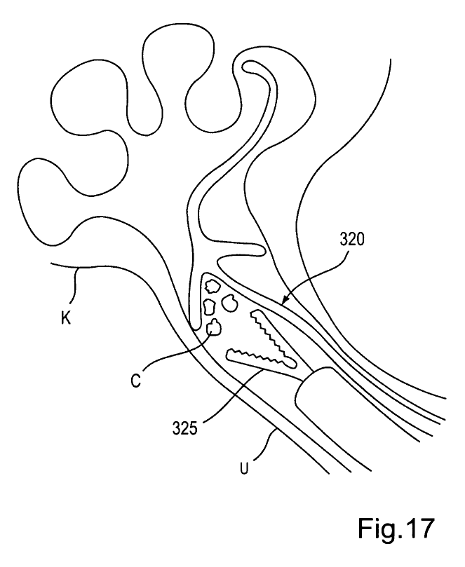

FIG. 17 is a side view of a portion of the medical device 320 shown in the expanded configuration and disposed within a ureter U near an opening of a kidney K. In this illustration, the medical device 320 is shown again being used as a backstop within the ureter U. A forceps device 325 is being used to collect and remove kidney stone chips C (i.e., portions of a kidney stone) from within the ureter U. The medical device 320 can also be used to sweep the kidney stone chips C from within the ureter U instead of, or in conjunction with, collecting the kidney stone chips C with the forceps device 325.

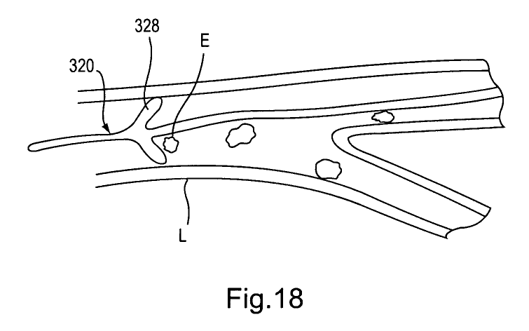

FIG. 18 illustrates another example use of the medical device 320. In FIG. 18, medical device 320 is shown in an expanded configuration and disposed within a vascular lumen L. In this example, the medical device 320 is being used as a backstop device during a procedure being performed within the vascular lumen L. The expandable member 328 can be used to prevent embolic particles E from migrating distally within the vascular lumen L.

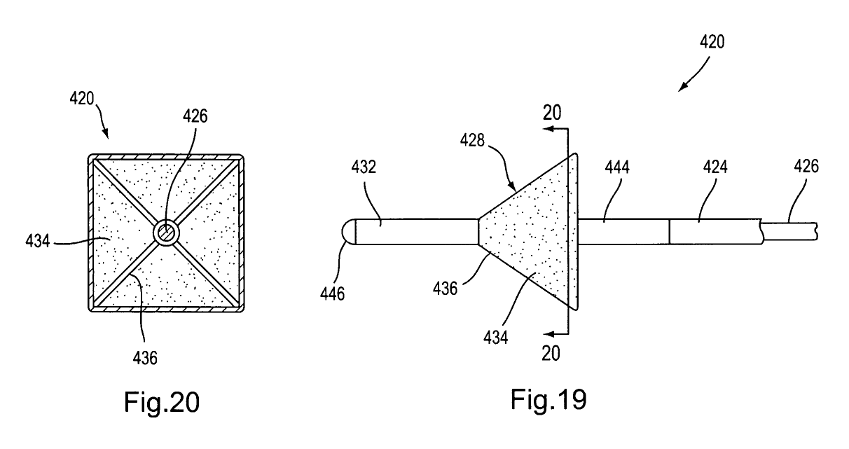

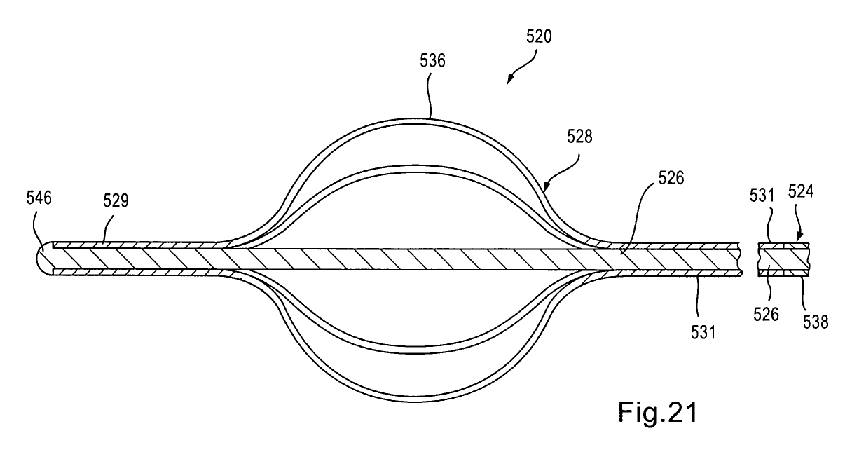

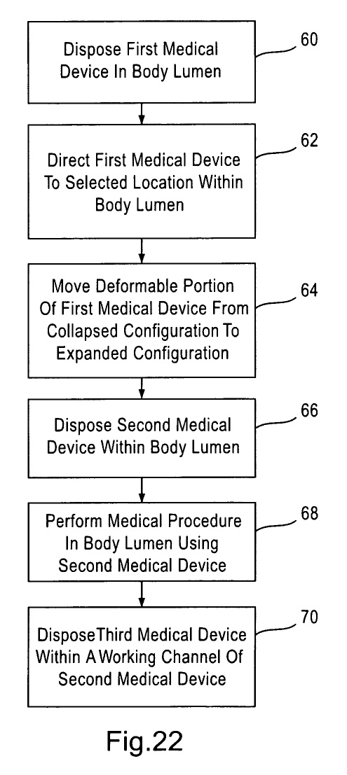

FIGS. 19 and 20 illustrate a portion of a medical device according to yet another embodiment of the invention, shown in an expanded configuration. A medical device 420 is similarly constructed as the previous embodiments, and can perform the same functions as previously described. The medical device 420 includes an elongate body 426 having a proximal end portion partially disposable within a lumen of an actuator 424. An expandable member 428 having a collapsed configuration (not shown) and an expanded configuration (FIGS. 21 and 22) is disposed at a distal portion of the elongate body 426. In this embodiment, the expandable member 428 includes four deformable portions 436, and a membrane 434 is coupled to the four deformable portions 436. The four deformable portions 436 can be formed from slits cut in a cylindrical tube as described above for medical device 120 or can be separate wire components as described for medical device 220.

The expandable member 428 defines a substantially square outer perimeter shape when the expandable member 428 is in the expanded configuration, as shown in the cross-sectional view of FIG. 20, and a substantially triangular side profile shape as shown in FIG. 19. As with the previous embodiments, the medical device 420 can optionally include one or more coil members (not shown), and the elongate body 426 can include a core portion (not shown), and a distal tip 446. The actuator 424 can move the expandable member 428 between the collapsed configuration and the expanded configuration as previously described.

FIG. 21 illustrates a medical device according to another embodiment of the invention. In this embodiment, a medical device 520 includes an elongate body 526, an actuator 524, and an expandable member 528. As shown in FIG. 21, this embodiment is an example of a medical device that does not include coil members coupled to the elongate body and/or expandable member and does not include cover members (e.g., cover member 132, 130, 232, 244, etc.). For illustration purposes, the medical device 520 is shown without a membrane coupled to the expandable member 528, but it is to be understood that a membrane can be included in this embodiment in the same manner as in previous embodiments. In this embodiment, the expandable member 528 has a collapsed configuration (not shown) and an expanded configuration as shown in FIG. 21. The expandable member 528 has a tubular form that defines multiple deformable portions 536 similar to the expandable member 128 of FIGS. 2 and 3. In this embodiment, a distal end portion 529 of the expandable member 528 is coupled to a distal tip 546 of the elongate body 526 and a proximal end portion 531 is coupled to or can be engaged by the actuator 524. As mentioned above for other embodiments, a length of the distal end portion 529 and a length of the proximal end portion 531 can vary from the lengths illustrated in FIG. 21.

The medical device 526 can be used for the same functions as described above for previous embodiments and the expandable member 528 can be actuated between the collapsed configuration and the expanded configuration in a similar manner as previously described. For example, to move the expandable member 528 from the collapsed configuration to the expanded configuration, the actuator 524 can be moved distally over a portion of the elongate body 526 while holding the elongate body 526 stationary such that a distal end 538 of the actuator 524 imparts a force on the proximal end 531 of the expandable member 528, causing the expandable member 528 to be moved to the expanded configuration.

FIG. 22 is a flow chart illustrating a method according to an embodiment of the invention. A method includes at 60, disposing a first medical device into a body lumen of a patient. The body lumen can be, for example, a ureter or a vascular lumen. The first medical device includes a expandable portion including a portion formed with a material resistant to energy emitted from a laser, and the expandable portion has a collapsed configuration and an expanded configuration. The expandable portion of the first medical device can be in the collapsed configuration when the first medical device is inserted into the body lumen of the patient. At 62, the first medical device can be directed to a selected location within the body lumen of the patient

At 64, the expandable portion is moved from the collapsed configuration to the expanded configuration while the first medical device is disposed within the body lumen of the patient. After disposing the first medical device within the body lumen, a second medical device is disposed into the body lumen of the patient at 66. At 68, a medical procedure is performed within the body lumen of the patient using the second medical device. The medical procedure can be, for example, a lithotripsy procedure in a ureter or a procedure to break-down emboli in a vascular lumen. The expandable portion, while in the expanded configuration, is configured to prevent distal migration of biological material within the body lumen of the patient during the medical procedure being performed by the second medical device.

In some embodiments, the second medical device defines a working/guidewire channel. In such an embodiment, when the second medical device is disposed within the body lumen, the working/guidewire channel of the second medical device is placed over the first medical device and moved distally along the first medical device. At 70, in some embodiments, a third medical device can be disposed within the working/guidewire channel of the second medical device and moved distally within the working/guidewire channel of the second medical device. In some embodiments, the second medical device can include two or more channels such that the first medical device can be disposed within one channel and a third medical device can be disposed within a separate channel.

The various components of the medical device 20 (120, 220, 320, 420) can each be constructed with a variety of different materials, such as biocompatible plastics and/or metals. For example, the components of the elongate body, the actuator and the expandable member can be formed with 400 series stainless steel, Nitinol, or a polymer such as Nylon, HDPE, Nano-Clays, etc. They may be formed with a single material or may be formed, for example by extrusion, of two or more materials. For example, in one embodiment, one portion of the elongate body is formed from a first material having a first durometer and another portion is formed from a second material having a second durometer. Accordingly, one portion of the elongate body (or any of the other components) may be made of a softer or more flexible material than that of the another portion. The cover members and distal tip can also be formed with biocompatible plastics and/or metals, such as, for example, a PTFE material, or hydrophilically coated polyurethane.

The membrane 34 (134, 234, 334, 434) can be formed with a variety of different biocompatible materials. For example, the membrane can be formed with a spun polycarbonate or polyurethane material, or with a silicone, EPTFE, PTFE, or other flouropolymer, or other material that provides protection against damage that can be caused by laser energy, produced by, for example, a laser lithotripsy device. For example, suitable materials may be formed with a light color material such that laser energy can be reflected. Such materials may also be formed, for example, with loose fibers that can move or separate as laser energy is directed at the material, and/or can absorb the laser energy.

### Conclusion

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents.

The previous description of the embodiments is provided to enable a person skilled in the art to make and/or use the invention. While the invention has been particularly shown and described with reference to embodiments thereof, it will be understood by those skilled in art that various changes in form and details may be made therein without departing from the spirit and scope of the invention. For example, the various features of a medical device 20 (120, 220, 320, 420, 520) may include other configurations, shapes and materials not specifically illustrated, while still remaining within the scope of the invention.

Various combinations and sub-combinations of the components described herein can be included in an embodiment of a medical device according to the invention. For example, a medical device according to the invention may or may not include an actuator having a tubular form, and may or may not include one or more cover members. Depending on the use for a medical device according to the invention, the medical device may or may not include a membrane. Further, in any of the various embodiments described herein, the expandable member can be formed from a tubular membrane, or from one or more wire portions coupled to an elongate body, or from some other suitable deformable member. Moreover, in any of the embodiments described herein, one or more coil members may or may not be included.

What is claimed is:

1. An apparatus, comprising:
an elongate body disposable within a body lumen of a patient; and
an expandable member coupled to the elongate body, the expandable member having a collapsed configuration and an expanded configuration, the expandable member when in the expanded configuration configured to substantially prevent distal migration of biological material within the body lumen during a medical procedure being performed in the body lumen using a medical device different than the elongate body.

2. The apparatus of claim 1, wherein the expandable member includes a plurality of deformable portions, the medical device further comprising:
a membrane disposed to cover the plurality of deformable portions.

3. The apparatus of claim 1, wherein the body lumen is a ureter.

4. The apparatus of claim 1, wherein the body lumen is a vascular lumen.

5. The apparatus of claim 1, wherein the medical procedure is a lithotripsy procedure in a ureter of the patient.

6. The apparatus of claim 1, wherein the medical procedure is a vascular procedure and the biological material includes a blood clot.

7. The apparatus of claim 1, further comprising:
an actuator coupled to the elongate body and configured to move the expandable member between the collapsed configuration and the expanded configuration.

8. The apparatus of claim 1, wherein the expandable member includes a plurality of wire members coupled to the elongate body.

9. The apparatus of claim 1, further comprising a cover member coupled to a distal end portion of the elongate body.

10. The apparatus of claim 1, further comprising:
a membrane coupled to the expandable member formed with a material resistant to energy emitted by a laser.

11. The apparatus of claim 1, further comprising:
a cannula coupled to the elongate body, the cannula including the expandable member.

12. An apparatus, comprising:
an expandable member having a collapsed configuration and an expanded configuration and configured to be disposed within a body lumen; and
a membrane coupled to the expandable member, the membrane configured and formulated to maintain adequate structural integrity when exposed to laser energy applied during a lithotripsy procedure performed within the body lumen when the expandable member is disposed within the body lumen and the expandable member is in the expanded configuration.

13. The apparatus of claim 12, further comprising:
an actuator operatively coupled to the expandable member, the actuator configured to move the expandable member between the collapsed configuration and the expanded configuration while disposed within the body lumen of the patient.

14. The apparatus of claim 12, further comprising:
an elongate body coupled to the expandable member; and
an actuator defining a lumen, the elongate body movably disposable within the lumen of the actuator.

15. The apparatus of claim 12, wherein the expandable member while in the expanded configuration is configured to substantially prevent distal migration of biological material within the body lumen during a medical procedure performed in the body lumen with a medical device other than the expandable member.

16. The apparatus of claim 12, wherein the expandable member while in the expanded configuration is configured to anchor the medical device within the body lumen of the patient.

17. The apparatus of claim 12, wherein the membrane is formed with at least one of a polycarbonate material, a polyurethane material, or a flouropolymer material.

18. A method, comprising:
disposing a first medical device into a body lumen of a patient, the first medical device having an expandable portion including a portion formed of a material resistant to energy emitted by a laser, the expandable portion having a collapsed configuration and an expanded configuration;
moving the expandable portion from the collapsed configuration to the expanded configuration while the first medical device is disposed within the body lumen of the patient;
after the disposing the first medical device, disposing a second medical device into the body lumen of the patient; and
performing a medical procedure within the body lumen of the patient with the second medical device, the expandable portion while in the expanded configuration configured to prevent distal migration of biological material within the body lumen of the patient during the medical procedure.

19. The method of claim 18, further comprising:
during the disposing the first medical device, directing the first medical device to a selected location within the body lumen of the patient.

20. The method of claim 18, wherein the expandable member of the first medical device is in the collapsed configuration during the disposing the first medical device.

21. The method of claim 18, wherein the body lumen is a ureter of the patient and the performing the medical procedure includes performing a lithotripsy procedure.

22. The method of claim 18, wherein the body lumen is a vascular lumen of the patient.

23. The method of claim 18, wherein the first medical device includes a first cannula coupled to an elongate body and the expandable member is disposed on the first cannula, and the moving the expandable member includes displacing a second cannula operatively coupled to the first cannula.

24. The method of claim 18 wherein the first medical device includes an elongate body and the expandable member is coupled to the elongate body, the moving the expandable member includes displacing a cannula operatively coupled to the elongate body.

25. The method of claim 18, wherein the second medical device defines a working channel and a guide wire channel, the disposing the second medical device includes:
disposing the guide wire channel of the second medical device over the first medical device; and
moving the second medical device distally along the first medical device.

26. The method of claim 18, further comprising:
disposing a third medical device within the working channel of the second medical device; and
moving the third medical device distally within the working channel of the medical device.

27. An apparatus, comprising:
an expandable member configured to be disposed within a body lumen, the expandable member defining a lumen and including a deformable portion, the expandable member having a collapsed configuration and an expanded configuration;
a membrane coupled to and substantially covering the deformable portion, the membrane being configured and formulated to maintain adequate structural integrity when exposed to laser energy; and
an actuator operatively coupled to the expandable member, the actuator configured to move the expandable member between the collapsed configuration and the expanded configuration.

28. The apparatus of claim 27, wherein the expandable member defines a plurality of slits disposed longitudinally along the expandable member, the slits being in communication with the lumen of the expandable member.

29. The apparatus of claim 27, wherein the actuator defines a lumen, the apparatus further comprising:
an elongate body movably disposable within the lumen of the actuator and the lumen of the expandable member.

30. The apparatus of claim 27, wherein the body lumen is a ureter.

31. The apparatus of claim 27, wherein the body lumen is a vascular lumen.

32. The apparatus of claim 27, further comprising:
an elongate body movably disposable within the lumen of the expandable member; and
a cover member coupled to a distal portion of at least one of the expandable member or the elongate body.
